(19) United States
(12) Patent Application Publication
(10) Pub. No.: US 2010/0280112 A1
(43) Pub. Date: Nov. 4, 2010

(54) Composition Enriched in Diglyceride with Conjugated Linoleic Acid

(75) Inventors: Untaek Lee, Seoul (KR); Jeeyoung Kim, Seoul (KR); Moonwon Lee, Kimpo (KR); Sunyoung Choi, Incheon (KR)

Correspondence Address: FISH & RICHARDSON P.C., PO BOX 1022, MINNEAPOLIS, MN, 55440-1022, US

(73) Assignee: KIP BIOTECH LLC, Minnetonka, MN (US)

(21) Appl. No.: 12/835,973

(22) Filed: Jul. 14, 2010

Related U.S. Application Data
(63) Continuation of application No. 10/580,947, filed on Aug. 7, 2006.

(30) Foreign Application Priority Data
Nov. 28, 2003 (KR) 10-2003-0085422
Nov. 26, 2004 (KR) 10-2004-0097924
Nov. 26, 2004 (KR) PCT/KR04/03083

(51) Int. Cl. A61K 31/231; A61P 35/00

(52) U.S. Cl. 514/547

(57) ABSTRACT

The present invention relates to an oil composition containing a large amount of a. diglyceride of conjugated linoleic acid (CLA), and more particularly to an oil composition with body weight control, anticancer, antioxidation and immune enhancement functions, which is based on a diglyceride of conjugated linoleic acid resulted from an enzymatic reaction between conjugated linoleic acid obtained from edible oil and glycerol. The inventive oil composition comprises 40-95% by weight of diglycerides, 5-60% by weight of triglycerides, 0.110% by weight of monoglycerides, and 0.02-10% by weight of residues, in which the ratio of conjugated linoleic acid (CLA) to fatty acids contained in the total glycerides is 5-98%. The use of the oil composition provides high-added-value foods, pharmaceutical compositions and foodstuff additives with effects of anticancer, immune enhancement, antioxidation, antichlorestol, growth promotion and the like.

### CLAIM OF PRIORITY

This application is a continuation of U.S. patent application Ser. No. 10/580,947, filed Aug. 7, 2006, which claims priority to PCT International Application No. PCT/KR04/03083, filed Nov. 26, 2004, which claims priority to Korean Application No. 10-2004-0097924, filed Nov. 26, 2004, which claims priority to Korean Application No. 10-2003-0085422, filed Nov. 28, 2003, all of which are incorporated by reference.

### TECHNICAL FIELD

The present invention relates to an oil composition containing a large amount of a diglyceride of conjugated linoleic acid, and more particularly to an oil composition with body weight control, anticancer, antioxidation and immune enhancement functions, which is based on a diglyceride of conjugated linoleic acid resulted from an enzymatic reaction between conjugated linoleic acids obtained from edible oil and glycerol.

### BACKGROUND ART

Conjugated linoleic acid (hereinafter, also referred to as “CLA”) is a generic term referring to a group of positional and geometric isomers of linoleic acid with conjugated double bonds in the cis or trans configuration.

CLA exists in the form of various isomers, including cis7, trans9-CLA; trans7, trans9-CLA; cis8, trans10-CLA; trans8, trans10-CLA; cis9, trans11-CLA; trans 9, trans11-CLA; cis10, trans12-CLA, trans10, trans12-CLA; cis 11, trans13-CLA; trans11, trans13-CLA. The most predominant CLA isomer in natural food is the cis9, trans11-CLA isomer, and a mixture of CLA synthesized from edible oil rich in linoleic acid mainly contains the cis9, trans 1-CLA and trans10, cis12-CLA isomers.

In many reports and literatures, the nutritional and physiological importance of CLA is found. CLA derived from animals with rumen is a natural multifunctional fatty acid which is known to have an inhibition or mitigation effect against skin cancer, stomach cancer, breast cancer and colon cancer by antimutagenic activity (Ha, et al., Cancer Res., 50:1097 (1990), Birt, et al., Cancer Res., 52:2035 (1992)), a therapeutic effect against diabetes by a reduction in glucose resistance, an inhibitory effect against fatness by a reduction in body fat (Cook et al, U.S. Pat. No. 5,554,646), and a prevention or control effect on high blood pressure, as well as various activities beneficial to the human body, such as immune enhancement (Cook et al, U.S. Pat. No. 5,674,901), antioxidation and anticholesterol (Nicolosi et al, Circulation 88(suppl.):2458, 1993), and antimold activities.

Currently, CLAs are used in the form of free fatty acids, ester derivatives, or triglycerides. CLAs in the form of free fatty acids are known to have problems in that they have toxicity at the terminal carboxylic group and are acidified fast, their preference are reduced due to unique taste and odor upon ingestion, and their use is limited to capsule-like products since when they are added to animal/vegetable oil, the quality of the oil can be caused to be bad.

For the CLA ester derivatives, there is a known method of preparing them by binding various functional substances, such as phospholipid and ascorbic acid, to CLA, as described in Korean Patent No. 0037151. However, the verification of their physiological function and product applicability is yet insufficient. Furthermore, CLAs processed in the form of free fatty acids or ester derivatives have problems in that oxidation resistance and processability are low, and irritating odor and taste are present, thus making it difficult to apply them for products.

Meanwhile, as it is known that CLA after ingestion is absorbed in vivo in the form of glycerides, it was expected that CLA products in form of glycerides would be more advantageous in terms of not only in vivo absorption rate after ingestion and but also application for foods and medicines, as compared to the above-described CLA products in the form of free fatty acids or ester derivatives.

In association with this, PCT publication No. WO 00/18994 discloses a composition comprising CLA in the form of triglyceride, and U.S. Pat. No. 6,609,222 discloses a composition comprising CLA and L-carnitine or its derivative in castor oil. In addition, PCT publication No. WO 03/043972 discloses a composition comprising, at the fatty acid positions of glycerides, a medium chain fatty acid including CLA, a long chain fatty acid, an ω-3 fatty acid, an ω-6 fatty acid, and an ω-9 fatty acid.

In addition, various patents disclose various methods to use the functionality of CLA, but relate mostly to triglycerides of CLA.

However, it is known that when triglyceride is orally ingested and digested in the gastro-intestine, two of three fatty acids of the triglyceride will be disassembled by lipase secreted from the pancreases and then absorbed into the intestines, but after absorption in vivo, will be bound in vivo again to form triglyceride, and flow in blood while they will be accumulated around the intestines or in subcutaneous fats.

Meanwhile, diglyceride is generally found in natural oil at a very small amount, and thus, has not been used for the general purpose although used in expensive cosmetics or medicines. However, due to the above-described problems of triglycerides, people generally try to use diglyceride (one form of glycerides) in foods, etc, actively these days.

This is because, unlike triglyceride, diglyceride is structurally stable while it is disassembled by lipase and not reassembled after in vivo absorption, so that it is completely combusted into water and carbon dioxide in the liver and muscles without in vivo accumulation.

Thus, U.S. Pat. No. 6,004,611 discloses diglyceride products which have the above-described characteristics and can be used for the general purpose. In addition, EP No. 1,135,991 describes the preparation of diglyceride with ω-3 fatty acids, and Japanese Patent No. 8269478 describes the preparation of diglyceride with medium chain fatty acid. Diglycerides for preventing the accumulation of fat in vivo as described above are now mainly applied in edible oil products, and already commercialized and now marketed in Japan and USA.

In view of such problems, the present inventors believed that if CLA is fed in vivo in the form of diglyceride, it could further enhance a preventive effect on the accumulation of fat as compared to the conventional diglyceride products, make products have a variety of the nutritional and physiological advantages of CLA in addition to the structural stability of oil, thus providing superiority to the conventional products. Thus, departing from the prior way of ingesting CLA in the form of triglyceride, the present inventors have attempted to develop an oil composition containing a diglyceride of CLA as a main ingredient and an application method thereof, and after long-term studies thereon, completed this development.

### DISCLOSURE OF INVENTION

In view of the above-mentioned problems occurring in the prior art, the present inventors believed that, if CLA is ingested in the form of diglyceride, it could further enhance a preventive effect on the accumulation of fat in vivo as compared to the conventional diglyceride products and provide to products a variety of the nutritional and physiological advantages of CLA in addition to the structural stability of oil, thus providing superiority to the conventional products. Thus, departing from the prior way of ingesting CLA in the form of triglyceride, the present inventors have attempted to develop an oil composition containing a diglyceride of CLA as a main ingredient and an application method thereof, and after long-term studies thereon, completed this development.

Accordingly, it is an object of the present invention to provide an oil composition containing a large amount of CLA in the form of diglyceride.

Another object of the present invention is to provide food containing the oil composition.

Still another object of the present invention is to provide functional foods and pharmaceutical compositions for body weight control, anticancer, antioxidation and immune enhancement, which contain the oil composition as an active ingredient.

To achieve the above objects, in one aspect, the present invention provides an oil composition comprising 40-95% by weight of diglycerides, 5-60% by weight of triglycerides, 0.1-10% by weight of monoglycerides, and 0.02-10% by weight of residues, in which the ratio of conjugated linoleic acid (CLA) to fatty acids contained in the total glycerides is 5-98%.

In another aspect, the present invention provides food containing the oil composition.

In still another aspect, the present invention provides functional foods and pharmaceutical compositions for body weight control, anticancer, antioxidation and immune enhancement, which contain the oil composition as an active ingredient.

The oil composition prepared by the present invention contains a mixture of free fatty acid and glycerol, which remain after a process of purifying diglyceride, a main component of the composition. The mixture is named “residues” herein. The residues are preferably contained at an amount of 0.02-10% by weight. This is because if the free fatty acid and the glycerol are completely removed from the composition, production costs will be increased. Thus, the composition is preferably used without complete removal of the free fatty acid and the glycerol.

Among fatty acids contained in the total glycerides, the conjugated linoleic acid (CLA) comprises at least one selected from the group consisting of cis-9, trans-11 CLA, trans-10, cis-12 CLA, and other CLA isomers.

However, the present invention is not limited to the above-described kinds of the generally known conjugated linoleic acid isomers.

In the present invention, the CLA is obtained from at least one edible oil selected from the group consisting of animal and vegetable oils rich in linoleic acid, including safflower oil, soybean oil, corn oil, rapeseed oil, rice bran oil, sesame oil, perilla oil, sunflower oil, cottonseed oil, peanut oil, olive oil, palm oil, palm olein oil, palm stearin oil, palm kernel oil, coconut oil, beef tallow, lard oil, mixed vegetable oil, shortening, margarine, pepper seed oil, Kapok oil, and Nica oil. Preferably, the CLA is obtained from vegetable oils, including safflower oil, corn oil, evening primrose oil, and sunflower oil.

Also, the oil composition of the present invention is used to produce general edible oil, salad oil, frying oil, margarine, fat spread, shortening, ice cream, whipped cream substitutes, dressings, Mayonnaise, and oil for confectionery, and the like.

Furthermore, the inventive oil composition is used in functional foods and pharmaceutical composition for body weight control, anticancer, antioxidation and immune enhancement, as an active ingredient.

Hereinafter, the present invention will be described in detail.

To prepare the oil composition of the present invention, CLA is first obtained from linoleic acid or linoleic acid-rich edible oils, such as safflower oil, corn oil, evening primrose oil, and sunflower oil, by conventional CLA synthesis methods (aqueous alkali isomerization, non-aqueous alkali isomerization, and alkali alcoholate isomerization.

Then, the obtained CLA is mixed with glycerol and subjected to enzymatic reaction with lypozyme RM IM in vacuum, to obtain a crude oil composition containing a large amount of CLA diglyceride. Then, fatty acids and monoglyceride are separated by fractional distillation, and the remaining material is refined by a conventional oil purification method.

The inventive oil composition thus obtained comprises more than 40% by weight of diglycerides, 5-60% by weight of triglycerides, less than 10% by weight of monoglycerides, and residues, i.e., less than 5% by weight of free fatty acid and less than 5% by weight of glycerol, in which the ratio of CLA to fatty acids contained in the total glycerides is more than 5%, and the remaining fatty acids consist of saturated fatty acids and unsaturated fatty acids with 4-22 carbon atoms.

In the present invention, an oil composition enriched in diglyceride, which has a ratio of CLA to total fatty acids of 5-20%, may be obtained by preparing a CLA-containing fatty acid composition from palm stearin or olive oil with a low content of linoleic acid and then reacting it lypozyme, a glyceride synthesis enzyme. Alternatively, an oil composition enriched in diglyceride, which has a ratio of CLA to total fatty acids of 50-95%, may be obtained by preparing a CLA-containing fatty acid composition from safflower oil or corn oil with a high content of linoleic acid and then reacting it lypozyme.

The inventive oil composition has little or no difference from the generally used conventional edible oils or shortenings in the physical and physical properties, and may be used as substitute foods for the prior edibles or shortenings, so that it can provide foods added with the known physiological effects of CLA.

For example, the inventive oil composition may be used in edible oil products for frying and cooking, dressing products such French dressings as water-in-oil or oil-in-water foods, Mayonnaise products, cream products, confectionery products such as chocolates and potato chips, drink products, capsule products, tablet products, powder products, bread products such as breads or cookies, and the like.

The use of the inventive oil composition is not limited to the above-described examples, and possible in all products containing the inventive oil composition. The inventive oil composition may be applied at various concentrations and used alone or in a mixture with other animal/vegetable oils.

Furthermore, the inventive oil composition may also be used in medicines in the form of solids such as powders, particles, capsules, pills or tablets, or liquid such as dispersions or emulsions, without limitations. Also, it may be formulated with general additives such as disintegrants, binders and excipients, and drugs.

The inventive oil composition may be administered orally at a general daily dose of 1-4 g one time or several times a day. However, it is to be understood that the actual dose of the inventive oil composition should be determined in view of various factors, such as oral administration formulations, and the age, sex, body weight and disease severity of patients, and thus, the scope of the present invention is not limited in any way to the above dose.

In addition to the purpose of providing the multi-functionality of CLA using the inventive oil composition in the preparation of processed animal and vegetable foods (sausages, canned foods, etc.), the oil composition may also be used as a substitute for oils so as to improve storage stability, preference and emulsion stability. The amount of the oil composition added is not limited, and as the content of the inventive oil composition in fat, which is used as a substitute for the fat in the preparation of, for example, sausages, is gradually increased to 5%, 20% and 40%, the storage stability and preference of the sausages were improved. As described above, the inventive oil composition may be generally used for the purpose of improving the storage stability, preference and emulsion stability of processed animal and vegetable foods, without specific limitations in its use concentration.

In addition, the inventive oil composition may be added to feedstuff additives for chicken raising, pig raising, dairy farming and cattle raising, for purposes such as the prevention of body fat accumulation, the promotion of growth, the prevention of diseases, and the supply of nutrients, in a mixture with materials, such as organic nutrients and inorganic nutrients.

### BEST MODE FOR CARRYING OUT THE INVENTION

Hereinafter, the present invention will be described in detail by the following examples and test examples. It is to be understood, however, that these examples are given for a better understanding of the present invention and not construed to limit to the scope of the present invention.

### Test Example

### 1. Gas Chromatography for Analysis of Fatty Acid Composition

Fatty acid composition was analyzed under the following conditions: column: HP-INNOWAX (Agilent Co., USA); carrier gas: 2.1 ml/min; helium; oven temperature: 150-260° C.; and sample concentration: 25 g/l (methylene chloride solvent). As a detector, a flame ionization detector (FID) was used at 275° C.

### 2. Liquid Chromatography for Analysis of Glyceride Composition

Glyceride composition was analyzed under the following conditions: column: Supelcosil LC-Si, 5 μm, 25 cm (Aupelco Co., USA); mobile phase solvents: solvent A (70 benzene: 30 chloroform: 2 acetic acid) and solvent B (ethyl acetate); sample concentration: 1 mg/ml (chloroform solvent); and detector: evaporative light scattering detector (ELSD). In this case, column temperature was 82° C., and flow rate was 2.3 ml/min.

### 3. Measurement of Body Fat Content

After administering diets for 6 weeks, the body weights of rats of each test group were measured. Then, the rats were killed by cervical dislocation, and each of parts of the rats was dissected and homogeneously ground with a blender while adding about 3-fold volume of distilled water. The ground material was dried at 80° C., and fat from the dried material was extracted by a Soxlet method with a chloroform-methanol (2:1) solvent. After completion of the extraction, the solvent portion was collected and dried, and the weight of the remaining fat was measured.

### Example 1

900 g of safflower oil from L company, Korea, was added to an alkali-glycerol mixture, 1100 g, and isomerized with 250 g of calcium hydroxide with heating to a 150° C. under a nitrogen atmosphere, thus preparing CLA. After completion of the alkali isomerization, the reaction solution was extracted two times with 500 ml of hexane, and the organic solvent layer was washed three times with water and concentrated, thus obtaining free fatty acids. The composition of the free fatty acids was analyzed by the gas chromatography method described in Test Example above, and the analysis results are shown in Table 1 below.

TABLE 1

| Fatty acid | Content (%) |
|---|---|
| 16:0 | 7.4 |
| 18:0 | 2.7 |
| 18:1 | 9.7 |
| 18:2 (non-conjugated fatty acid) | 2.6 |
| Conjugated fatty acid (18:2) | 77.6 |
| (cis-9, trans-11) | (36.1) |
| (trans-10, cis-12) | (38.4) |
| (other isomers) | (3.1) |

Then, 283.7 g of the prepared CLA and 46.2 g of glycerol were mixed with each other, and added with 4.255 g of lipozyme RM IM (Novozyme). The mixture was allowed to react under a vacuum of 20 Torr at 40° C. for 10 hours with stirring at 300 rpm. The enzyme is removed through a filter, thus obtaining about 330 g of oil. Then, unreacted reactants were removed by molecular distillation, thus obtaining 300 g of oil containing triglyceride and diglyceride as main components. Then, a conventional purification process for decoloration and deodorization was performed, thus obtaining an oil composition according to the present invention.

The oil composition was analyzed for fatty acids and glycerides by the methods described in Test Example above, and the analysis results are shown in Tables 2 and 3 below.

### Example 2

1000 g of safflower oil obtained from 0 company, Korea, was dissolved in 750 g of water and hydrolyzed with lipase-OF under conditions of 200 rpm and 40° C., thus obtaining 900 g of fatty acids from the safflower oil.

283.7 g of the prepared fatty acids and 46.2 g of glycerol were mixed with each other and added with 4.225 g of Lipozyme RM IM. The mixture was allowed to react under a vacuum of 20 Torr at 40° C. for 10 hours with stirring at 300 rpm. Then, the enzyme was removed through a filter, thus obtaining 330 g of oil. Then, a purification process as described in Example 1 was performed, thus obtaining an oil composition.

The oil composition was analyzed as described in Example 1, and the analysis results are shown in Tables 2 and 3 below.

### Example 3

The oil composition prepared in Example 1 and the oil composition prepared in Example 2 were mixed at a weight ratio of 1:7, thus obtaining an oil composition.

Then, the oil composition was analyzed for fatty acids and glycerides as described in Examples 1 and 2, and the analysis results are shown in Tables 2 and 3 below.

### Comparative Example

A safflower product commercially available from 0 company, Korea, was used for comparison with Examples 1, 2 and 3.

TABLE 2

| | Content (%) | | | |
|---|---|---|---|---|
| | Exam- | Exam- | Exam- | Comparative |
| Fatty acid | ple 1 | ple 2 | ple 3 | Example |
| 16:0 | 6.1 | 7.0 | 6.5 | 7.4 |
| 18:0 | 2.3 | 3.0 | 2.9 | 2.9 |
| 18:1 | 11.6 | 15.6 | 15.3 | 15.3 |
| 18:1 (non-conjugated) | 2.4 | 74.4 | 65.2 | 74.4 |
| Conjugated linoleic acid 18:2 | 77.6 | 0 | 10.1 | 0 |
| (cis-9, trans-11) | (34.1) | 0 | (4.6) | 0 |
| (trans-10, cis-12) | (36.4) | 0 | (4.8) | 0 |
| (other isomers) | (7.1) | 0 | (0.7) | 0 |

TABLE 3

| | Content (%) | | | |
|---|---|---|---|---|
| | | | | Comparative |
| Component | Example 1 | Example 2 | Example 3 | Example |
| Triglyceride | 16.3 | 15.7 | 15.9 | 98.7 |
| Diglyceride | 83.2 | 83.5 | 83.4 | 1.0 |
| Monoglyceride | 0.3 | 0.5 | 0.5 | 0 |
| Free fatty acid | 0.2 | 0.3 | 0.3 | 0.3 |

### Example 4

### Inhibitory Effect Against Increase of Body Weight

The CLA-containing oil composition prepared in the present invention was administered to test animals in order to examine if the composition has inhibitory effects against the increase of body weight and body fat.

Test animals were divided into groups administered with the compositions of Examples 1-3 and a group administered with the composition of Comparative Examples, each group consisting of 10 six-week-old SD-rats.

Each of the compositions was administered orally to each animal at an amount of 50 mg/kg one time a day in addition to feedstuffs. Then, the body weight of each animal was measured for each period according to the method described in Test Example 3. The mean value of the measured body weights is shown in Table 4 below.

TABLE 4

| | Body weight (g) | | | |
|---|---|---|---|---|
| | | | | Group |
| | | | | administered |
| | Group | Group | Group | with |
| Administration | administered | administered | administered | composition of |
| period of | with composition | with composition | with composition | Comparative |
| feedstuff | of Example 1 | of Example 2 | of Example 3 | Example |
| Day 0 | 139.5 ± 3.8 | 138.4 ± 4.7 | 139.8 ± 3.5 | 140.0 ± 3.1 |
| Day 14 | 275.7 ± 7.9 | 279.8 ± 6.5 | 277.8 ± 7.5 | 295.3 ± 4.5 |
| Day 21 | 343.2 ± 10.1 | 347.6 ± 9.3 | 345.4 ± 10.9 | 358.6 ± 11.7 |
| Day 42 | 369.2 ± 10.9 | 381.5 ± 9.5 | 375.3 ± 11.3 | 401.8 ± 11.9 |
| Body fat | 30.6 ± 2.9 | 61.4 ± 4.5 | 44.2 ± 3.9 | 97.44 ± 6.4 |
| content (g) | | | | |
| Body fat | 8.5 ± 0.8 | 16.4 ± 1.7 | 11.4 ± 1.6 | 24.7 ± 2.8 |
| content (%) | | | | |

As can be seen in Table 4, it could be found that the groups administered with the composition of Examples 1, 2 and 3, respectively, which contain a high concentration of diglyceride, showed the tendency of a decrease in body weight increase rate as compared to the group administered with the composition of Comparative Example, and were significantly lower in body fat content. Also, the groups administered with the compositions of Example 1 and Example 3 containing a high concentration of a diglyceride of CLA showed a tendency to inhibit the increase of body weight and could significantly inhibit the increase of body fat, as compared to the group administered with the composition of Example 2 containing general diglyceride.

### Example 5

### Preparation of Feedstuff with Inventive Oil Composition

Feedstuffs having compositions given in Table 5 below were administered to test animals. In this case, as the liquid oil component, each of the compositions of Examples 1-3 and Comparative Example was used for each test animal group. As the test animals, SD-rats were used.

TABLE 5

| Feedstuff components | Content (wt %) |
|---|---|
| Liquid oil | 10 |
| Casein | 20 |
| Minerals | 3.5 |
| Vitamins | 1.0 |
| DL-methionine | 0.3 |
| Potato starch | 60.2 |
| Cellulose | 5.0 |
| Total | 100 |

During the administration of the feedstuffs, all the test animals were confirmed to ingest the feedstuffs without rejection.

Accordingly, the inventive composition may be used in feedstuffs, so that it is expected that the good quality feedstuffs enriched in CLA and diglyceride can be provided by containing the compositions.

### Example 6

### Fried Food with Inventive Oil Composition

Each of the oil compositions of Examples 1-3 and Comparative Example was used to fry 50 g of frozen potatoes, thus preparing 10 fried potatoes for each composition. Then, the tastes of the fried food, odor in cooking, the mouth feel of the fried food, sputtering in cooking, and oxidation stability, were compared between the compositions. In such sensory tests, the taste, odor and mouth feel of the fried food were evaluated by sensory panels according to the method in Jang Kun-Hyung, Sensory Evaluation of Food Preference, Gaemunsa Co., 1975.

As can be seen in Table 6 below, the evaluation results showed the compositions of Examples 1, 2 and 3, which are enriched in diglyceride, were excellent in the taste and mouth feel of the fried food as compared to the oil composition of Comparative Example, a triglyceride product, and sputtering and odor in cooking were similar in all the oil compositions tested.

TABLE 6

| | | | | Comparative |
|---|---|---|---|---|
| | Example 1 | Example 2 | Example 3 | Example |
| Taste | 4.0 | 4.5 | 4.8 | 3.8 |
| Mouth feel | 4.6 | 4.6 | 4.6 | 4.3 |
| Odor | 4.0 | 4.0 | 4.0 | 4.0 |

Evaluation criteria:
5: very good,
4: good,
3: moderate,
2: bad,
1: very bad.

Also, in the measurement of color values before and after frying, by a Lovibond method which is conventionally used in color value measurement, glass color filters with different concentrations were compared with each other while the number of a filter corresponding to the color of the sample was read and expressed as total color value. In acid value measurement, a suitable amount of the sample was dissolved in 20 ml of an ether and ethanol (1:1) solvent, and the solution was added with 1% phenolphthalein and titrated with 0.1N potassium hydroxide. Also, oxidation induction time was measured with Metrohm 743 Rancimat in an aeration condition of 20 l/hr at 120° C. The measurement results are shown in Table 7 below.

TABLE 7

| | | | | Comparative |
|---|---|---|---|---|
| | Example 1 | Example 2 | Example 3 | Example |
| Change (%) in | 78 | 88 | 87 | 85 |
| acid value before | | | | |
| and after frying* | | | | |
| Change (%) in | 82 | 98 | 91 | 94 |
| color value before | | | | |
| and after frying** | | | | |
| Oxidation induction | 4.35 | 3.04 | 3.85 | 3.14 |
| time (hr) | | | | |

*change (%) in acid value before and after frying calculated based on acid value before frying.
**change (%) in total color value before and after frying calculated based on total color value (10 × R + Y).

The results showed that the compositions of Examples 2 and 3 were longer in oxidation induction time than the composition of Comparative Example. This suggests that the compositions of Examples 1 and 2 are chemically and physically stable.

### Example 7

### Preparation of Oil-in-Water Food

Mayonnaise comprising 80 wt % of the oil composition of Example 1, 7% of the egg yolk, 9 wt % of vinegar, 2 wt % of sugar, 0.5 wt % of mustard and 0.5 wt % of pepper was prepared by a conventional method. Also, another mayonnaise having the same composition as the above mayonnaise except for the composition of Example 3 was prepared. As a control group, the conventional mayonnaise (Ottogi Co., Korea) was used for the comparison of emulsion stability.

In emulsion stability test, mayonnaise was put in a scaled test tube, and shaked in a constant temperature water bath at 85° C. for 5 hours. Then, the mayonnaise was left to stand at room temperature and the amount of oil separated was measured and expressed as a percentage based on the total mayonnaise volume. The test results are shown in Table 8 below. As can be seen in Table 8, the mayonnaise containing the inventive oil composition showed emulsion stability similar to the prior mayonnaise.

TABLE 8

| | Amount of |
|---|---|
| | separated |
| | oil (%) |
| Mayonnaise containing composition of Example 1 | 32 |
| Mayonnaise containing composition of Example 3 | 35 |
| Control group (Product M from O company) | 36 |

### Example 8

### Preparation of Water-in-Oil Food

35.0 wt % of the oil composition of Example 1, 45.0% of hydrogenated soybean oil (IV=43), 0.7 wt % of natural cream flavor, 0.4 wt % of lecithin, 0.06 wt % of oil-soluble vitamin, 16.0 wt % of water, 2.5 wt % of skimmed milk powder, 0.3 wt % of salt and 0.04 wt % of sodium dehydroacetate were mixed by a homomixer, thus preparing margarine. Also, another margarine having the same composition except for the oil composition of Example 3 was prepared.

Then, the emulsion stabilities of the prepared inventive margarines and the conventional margarine (vegetable margarine, Ottogi Co., Korea) were measured and compared with each other.

In the measurement of the emulsion stabilities, each of the margarines was stored at 15° C. for 7 days, put in a scaled test tube, and left to stand at 40° C. for 5 hours, and the amount of separated oil was measured and expressed as a percentage based on the total margarine volume. The test results are shown in Table 9 below. In the test results, the margarines containing the inventive oil composition showed no great difference in emulsion stability from the prior margarine.

TABLE 9

| | Amount of |
|---|---|
| | separated |
| | oil (%) |
| Margarine containing composition of Example 1 | 68 |
| Margarine containing composition of Example 1 | 66 |
| Control group (Product M of O company) | 64 |

### Example 9

### Preparation of Ice Cream

12 wt % of the oil composition of Example 1, 10 wt % of butter, 12 wt % of skimmed milk powder, 10 wt % of condensed milk, 6 wt % of sugar, 0.5 wt % of gelatin and 49.5 wt % of water were mixed and subjected to sterilization, aging and freezing processes, thus preparing an ice cream. Another ice cream having the same composition except for the composition of Example 3 was prepared.

Then, an ice cream prepared with general edible oil and the ice creams prepared as described above were compared with each other for mouth feel. In this case, the sensory evaluation of mouth feel was performed by 20 expert sensory panels. The evaluation results are shown in Table 10 below. As can be seen in Table 10, the ice creams containing the inventive oil composition has no difference in mouth feel from the prior ice cream.

| | Evaluation |
|---|---|
| | score |
| Ice cream with composition of Example 1 | 4.0 |
| Ice cream with composition of Example 3 | 4.5 |
| Control ice cream | 4.8 |

5: very excellent,
4: excellent,
3: good,
2: bad,
1: very bad

### Example 10

### Preparation of Pharmaceutical Composition

### 1. Tablets

The following components were formulated according to a tablet preparation method based on general formulation rules in Korean pharmacopoeia, thus preparing tablets containing 200 mg of the oil composition of Example 1 per tablet:

Oil composition of Example 1+starch: 400 mg

Magnesium stearate: 5 mg

Calcium carboxymethylcellulose: 25 mg

Light anhydrous silicic acid: 70 mg

Sum total: 500 mg

### 2. Soft Capsules

The following components were formulated according to a capsule preparation method based on general formulation rules in Korean pharmacopoeia, thus preparing tablets containing 500 mg of the oil composition Of Example 1 per capsule:

Oil composition of Example 1: 500 mg

Gelatin: 497 mg

Paraoxymethyl benzoate: 1.5 mg

Paraoxypropylmethyl benzoate: 1.5 mg

Sum total: 1,000 mg

### INDUSTRIAL APPLICABILITY

As described above, the present invention provides the oil composition containing a large amount of the CLA diglyceride obtained by reacting CLA having the effects of anticancer, immune enhancement, antioxidation, anticholesterol and growth promotion with glycerol so as to form the CLA diglyceride which is almost combusted without accumulation in vivo.

Also, the present invention provides the pharmaceutical compositions and functional foods, which contain the oil composition as an active ingredient, together with a pharmaceutically available carrier.

Thus, the present invention can provide foods and pharmaceutical compositions having the effects of anticancer, immune enhancement, antioxidation, anticholesterol, growth promotion and body weight control.

What is claimed is:

1. An oil composition comprising about 80-95% by weight of diglycerides, wherein about 50-95% of the fatty acids contained in the total glycerides are a conjugated linoleic acid (CLA).

2. The oil composition of claim 1, wherein the composition contains 0.01-5% by weight, based on the total weight of the composition, of free fatty acid and 0.01-5% by weight of glycerol.

3. The oil composition of claim 1, wherein the CLA is selected from the group consisting of cis-9, trans-11 CLA, trans-10, cis-12 CLA, and other CLA isomers.

4. The oil composition of claim 1, wherein the CLA is obtained from animal or vegetable oils enriched in linoleic acid selected from the group consisting of safflower oil, soybean oil, corn oil, rapeseed oil, rice bran oil, sesame oil, perilla oil, sunflower oil, cottonseed oil, peanut oil, olive oil, palm oil, palm olein oil, palm stearin oil, palm kernel oil, coconut oil, beef tallow, lard oil, mixed vegetable oil, shortening, margarine, pepper seed oil, Kapok oil, and Nica oil.

5. A food comprising the oil composition of claim 1.

6. The food of claim 5, wherein the food is selected from the group consisting of edible oil, salad oil, frying oil, margarine, fat spread, shortening, ice cream, whipped cream substitutes, dressings, Mayonnaise, and oil for confectionery.

7. A pharmaceutical composition comprising the oil composition of claim 1 as an active ingredient.

8. The pharmaceutical composition of claim 7, which is in a tablet, capsule, powder or liquid form.

9. A feedstuff additive comprising the oil composition of claim 1.

10. The oil composition of claim 1, wherein the CLA is selected from the group consisting of cis-7, trans-9 CLA, trans-7 CLA, cis-8, trans-10 CLA, trans-8, trans 10 CLA, trans-9, trans-11 CLA, cis-10, trans-12 CLA, trans-10, trans-12 CLA, cis-11, trans-13 CLA; and trans-11, trans-12 CLA.

11. The oil composition of claim 1, wherein at least about 77% of the fatty acids contained in the total glycerides are a CLA.
